(19) United States
(12) Patent Application Publication
(10) Pub. No.: US 2018/0221241 A1
(43) Pub. Date: Aug. 9, 2018

(54) ENHANCEMENT OF VASODILATORY FUNCTION AND LOWERING OF EFFECTIVE SYSTEMIC VASCULAR RESISTANCE

(71) Applicant: THE GOVERNORS OF THE UNIVERSITY OF ALBERTA, Edmonton (CA)

(72) Inventor: Michael Sean MCMURTRY, Edmonton (CA)

(21) Appl. No.: 15/580,212

(22) PCT Filed: Jun. 7, 2016
(86) PCT No.: PCT/CA2016/050647

§ 371 Date: Dec. 6, 2017

Related U.S. Application Data
(60) Provisional application No. 62/172,379, filed on Jun. 8, 2015.

(51) Int. Cl. A61H 31/02; A61P 9/00; A61K 33/00

(52) U.S. Cl. CPC A61H 31/02; A61P 9/00; A61K 33/00; A61H 2201/0184; A61H 2203/03; A61H 2201/5071; A61H 2230/208; A61H 2230/206; A61H 2201/107; A61H 2201/1238

(57) ABSTRACT

A method for treating, preventing, or ameliorating cardiovascular disease in a subject involves exposing the subject for an effective duration to a condition of hypobaric normoxia or hypobaric hyperoxia. An apparatus or system useful for treating, preventing, or ameliorating cardiovascular disease includes an airtight chamber configured to accommodate and enclose a subject, and including at least one hatch to allow entry or exit of the subject; a vacuum source for adjusting pressure within the chamber to a level sufficient to maintain hypobaria; and a gas source for introducing one or more gases into the chamber, such as oxygen in an amount sufficient to produce a condition of normoxia or hyperoxia within the chamber, while maintaining hypobaria.

### CROSS-REFERENCE TO RELATED APPLICATIONS

This application claims the benefit of U.S. Patent Application No. 62/172,379, filed Jun. 8, 2015, the disclosures of which are hereby incorporated by reference in their entirety.

### FIELD OF THE INVENTION

The present invention relates to apparatuses, systems, and methods and uses of the apparatuses and systems for treating, preventing, or ameliorating cardiovascular disease in a subject. More particularly, the invention relates to apparatuses, systems, and methods and uses of the apparatuses and systems for inducing hypobaric normoxia or hypobaric hyperoxia in subjects having cardiovascular disease.

### BACKGROUND OF THE INVENTION

The overall mortality impact of cardiovascular diseases is unequivocally tremendous worldwide. However, there are geographical patterns in the prevalence and incidence of certain cardiovascular diseases. In addition to spatial patterns, there may be elevation-related trends to the distribution of incidence and prevalence of cardiovascular pathologies. There are epidemiological reports of lower rates of myocardial infarction and death in humans living at higher elevations, but the mechanism is not known (Burtscher, 2014). Ambient air pressure is lower at higher elevations, and changes with elevation in magnitudes that might be physiologically important. Significantly improved outcomes have been noted in coronary artery disease patients residing at higher altitudes versus those at sea level. This observation retains validity even after the adjustment of a multitude of medical risk factors (i.e., hypertension, dyslipidemia, diabetes, smoking status), socioeconomic factors (i.e., house hold income, education status), and environmental factors (i.e., ultraviolet B light, fine particulate air pollution of PM2.5).

Apart from the human and anthropogenic factors mentioned above, a major factor affected by changes in elevation is barometric reading of the ambient atmosphere. This barometric pressure is almost inversely linear to altitude, dropping with meter increases in elevation from sea level. The structure and function of biological articles are highly susceptible to physical forces acting upon them; thus, blood vessels may be affected by changes in pressure both inside and outside the vessels.

At the level of arteries and arterioles, constriction and vasodilation are fairly well described. The nitric oxide pathway is the major vasodilator mechanism associated with response to increased blood flow (exerting sheer stress on the blood vessel). Apart from the nitric oxide pathway, prostaglandins act to dilate vessels in response to intrinsic molecules or to some extent, to sheer strain and stress. These vasodilators influence myocyte activity to induce smooth muscle relaxation and subsequent dilation of blood vessels. In the living mammal, the pressure and flow inside of a vessel are major mediators of myogenic tone. On this basis, flow-mediated vasodilation of the brachial artery is a powerful clinical tool to assess endothelial integrity in patients exhibiting compromised vascular parameters resulting from a multitude of factors (i.e., heart failure, hypertension, and atherosclerosis). However, the brachial artery is less important to overall systemic blood pressure flow regulation than some other peripheral arterial vasculature. Mesenteric blood vessels represent such a microvascular bed, highly capable of altering resistance to a variety of stimulators in order to regulate the blood flow it sees.

Elevation-related reductions in barometric pressure may affect the structure and function of human vasculature. The structure of a blood vessel is highly responsive to an array of endogenous vasoconstrictors and vasodilators, inspiring therapeutic practices of administrating vasoactive drugs to treat vascular pathologies. An intra-aortic balloon pump is a commonly used invasive mechanical device to increase myocardial oxygen perfusion and cardiac output simultaneously.

Accordingly, the need remains for an apparatus and method for treating cardiovascular disease without subjecting the patient to medications or invasive surgery.

### SUMMARY OF THE INVENTION

The present invention relates to apparatuses, systems, and methods and uses of the apparatuses and systems for treating, preventing, or ameliorating cardiovascular disease in a subject. More particularly, the invention relates to apparatuses, systems, and methods and uses of the apparatuses and systems for inducing hypobaric normoxia or hypobaric hyperoxia in subjects having cardiovascular disease.

In one aspect, the invention comprises a method for treating, preventing, or ameliorating cardiovascular disease in a subject comprising exposing the subject for an effective duration to a condition of either hypobaric normoxia or hypobaric hyperoxia.

In one embodiment, the method comprises enclosing the subject within an airtight chamber wherein pressure within the chamber is adjusted to maintain hypobaria. In one embodiment, the method comprises adjusting the pressure by extracting at least a portion of air within the chamber. In one embodiment, the pressure is adjusted to a pressure equivalent to the pressure encountered at an altitude between 1500 m and 3000 m above sea level. In one embodiment, the pressure is adjusted to be lower than ambient air pressure by at least about 10 mmHg. In one embodiment, the pressure is adjusted to be lower than ambient air pressure by at least about 250 mmHg. In one embodiment, the pressure is adjusted to be lower than ambient air pressure in an amount sufficient to produce a condition of normoxia within the chamber, while maintaining hypobaria.

In one embodiment, the method further comprises introducing air into the chamber to ventilate the chamber.

In one embodiment, the method further comprises introducing oxygen into the chamber to prevent hypoxia at the adjusted pressure. In one embodiment, the oxygen is introduced in an amount sufficient to produce a condition of normoxia within the chamber, while maintaining hypobaria. In one embodiment, the oxygen is introduced in an amount sufficient to produce a condition of hyperoxia within the chamber, while maintaining hypobaria. In one embodiment, the oxygen is in the form of pure oxygen or oxygen-enriched air.

In one embodiment, the cardiovascular disease is selected from coronary artery disease, cerebrovascular disease, or peripheral artery disease. In one embodiment, the cardiovascular disease is selected from myocardial infarction or stroke. In one embodiment, the effective duration is at least once daily for at least about one hour.

In another aspect, the invention comprises use of an apparatus or system for treating, preventing, or ameliorating cardiovascular disease, the apparatus or system comprising:

an airtight chamber configured to accommodate and enclose a subject, and comprising at least one hatch to allow entry or exit of the subject;

a vacuum source for adjusting pressure within the chamber to a level sufficient to maintain hypobaria; and

a gas source for introducing one or more gases into the chamber, wherein the one or more gases comprises oxygen in an amount sufficient to produce a condition of either normoxia or hyperoxia within the chamber, while maintaining hypobaria.

In one embodiment, the one or more gases comprise air for ventilating the chamber. In one embodiment, the pressure is adjusted to a pressure equivalent to the pressure encountered at an altitude between 1500 m and 3000 m above sea level. In one embodiment, the pressure is adjusted to be lower than ambient air pressure by at least about 10 mmHg. In one embodiment, the pressure is adjusted to be lower than ambient air pressure by at least about 250 mmHg. In one embodiment, the pressure is adjusted to be lower than ambient air pressure in an amount sufficient to produce a condition of normoxia within the chamber, while maintaining hypobaria. In one embodiment, the oxygen is in the form of pure oxygen or oxygen-enriched air.

In one embodiment, the apparatus or system is used to treat, prevent, or ameliorate coronary artery disease, cerebrovascular disease, or peripheral artery disease. In one embodiment, the apparatus or system is used to treat, prevent, or ameliorate myocardial infarction or stroke.

In yet another aspect, the invention comprises an apparatus or system for treating, preventing, or ameliorating cardiovascular disease, comprising:

an airtight chamber configured to accommodate and enclose a subject, and comprising at least one hatch to allow entry or exit of the subject;

a vacuum source for adjusting pressure within the chamber to a level sufficient to maintain hypobaria; and

a gas source for introducing one or more gases into the chamber, wherein the one or more gases comprises oxygen in an amount sufficient to produce a condition of either normoxia or hyperoxia within the chamber, while maintaining hypobaria.

In one embodiment, the apparatus or system further comprises an alarm system comprising one or more sensors and one or more alarms.

Additional aspects and advantages of the present invention will be apparent in view of the description, which follows. It should be understood, however, that the detailed description and the specific examples, while indicating preferred embodiments of the invention, are given by way of illustration only, since various changes and modifications within the scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1A is a graph showing the effect of perfusion pressure (674 mmHg, 714 mmHg, and 754 mmHg) upon the lumen diameter (μm) of a vessel immersed in a calcium-free solution to mimic “passive” vessel responses.

FIG. 1B is a graph showing the effect of perfusion pressure (674 mmHg, 714 mmHg, and 754 mmHg) upon the lumen diameter (μm) of a vessel immersed in a calcium-enriched solution to mimic actual physiological conditions or “active” vessel responses.

FIG. 2 is a graph showing the effect of step-wise increases in flow (μl/min) on the lumen diameter (μm) of a vessel.

FIG. 3A is a graph comparing the response (% vasodilation) of a vessel to a perfusion pressure of 754 mmHg in the presence and absence of inhibitors (meclofenamate and L-NAME) (*p<0.01 vs. control; x-axis denotes perfusion pressures (mmHg) maintained inside the vessel by the pressure transducer).

FIG. 3B is a graph comparing the response (% vasodilation) of a vessel to a perfusion pressure of 714 mmHg in the presence and absence of inhibitors (meclofenamate and L-NAME) (*p<0.01 vs. control; x-axis denotes perfusion pressures (mmHg) maintained inside the vessel by the pressure transducer).

FIG. 3C is a graph comparing the response (% vasodilation) of a vessel to a perfusion pressure of 674 mmHg in the presence and absence of inhibitors (meclofenamate and L-NAME) (*p<0.01 vs. control; x-axis denotes perfusion pressures (mmHg) maintained inside the vessel by the pressure transducer).

FIG. 4A is a graph showing the effect of a vasodilator (methylcholine cumulatively added in a dose range of 1 nM to 1 μm) on a vessel (% change in lumen diameter) in the absence of inhibitors (L-NAME and meclofenamate).

FIG. 4B is a graph showing the effect of a vasodilator (methylcholine cumulatively added in a dose range of 1 nM to 1 μm) on a vessel (% change in lumen diameter) in the presence of inhibitors (L-NAME and meclofenamate).

FIGS. 5A-C are graphs showing left ventricular pressure-volume relationships expressed as PV loops obtained via invasive catheterization.

FIGS. 6A-G are graphs showing the results of invasive pressure-volume hemodynamic analyses: systolic blood pressure (SBP) and diastolic blood pressure (DBP) (FIG. 6A); end-systolic pressure (ESP) (FIG. 6B); end-systolic volume (EDV) (FIG. 6C), maximum derivative of change in systolic pressure over time (dp/dt max) (FIG. 6D); stroke volume (SV) (FIG. 6E); cardiac output (CO) (FIG. 6F); and systemic total vascular resistance (STVR) (FIG. 6G).

FIGS. 7A and 7B are transthoracic images obtained on anesthetized control mice on Day 1 (immediately after left anterior descending artery (LAD)-ligation surgery) (FIG. 7A) and on Day 7 (FIG. 7B). M-mode images were captured using a parasternal short axis view.

FIGS. 8A and 8B are transthoracic images obtained on anesthetized low air pressure treatment mice on Day 1 (immediately after left anterior descending artery (LAD)-ligation surgery) (FIG. 8A) and on Day 7 (FIG. 8B). M-mode images were captured using a parasternal short axis view.

FIG. 9A is a graph showing the heart rate (beats per minute) on Days 1 and 7 of mice treated with low air pressure and untreated control mice.

FIG. 9B is a graph showing fractional shortening (%) on Days 1 and 7 of mice treated with low air pressure and untreated control mice.

FIG. 9C is a graph showing ejection fraction (%) on Days 1 and 7 of mice treated with low air pressure and untreated control mice.

FIG. 9D is a graph showing stroke volume (pi) on Days 1 and 7 of mice treated with low air pressure and untreated control mice.

FIG. 9E is a graph showing cardiac output (ml/min) on Days 1 and 7 of mice treated with low air pressure and untreated control mice.

FIGS. 10A-C are schematic diagrams indicating forces on an artery at atmospheric pressures of 760 mmHg (FIG. 10A), 714 mmHg (FIG. 10B), and 674 mmHg (FIG. 10C).

FIG. 11 is an image of one embodiment of a chamber of the present invention.

FIG. 12 is an image of one embodiment of a chamber of the present invention.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a,” “an,” and “the” include plural referents unless the context clearly dictates otherwise.

The present invention relates to apparatuses, systems, and methods and uses of the apparatuses and systems for treating, preventing, or ameliorating cardiovascular disease in a subject. More particularly, the invention relates to apparatuses, systems, and methods and uses of the apparatuses and systems for inducing hypobaric normoxia or hypobaric hyperoxia in subjects having cardiovascular disease in a non-invasive manner. As used herein, the term “non-invasive” means not requiring the introduction or entry of instruments (for example, surgical instruments) into the body of the subject, thereby avoiding damage to biological tissues. It was surprisingly discovered that induction of hypobaric normoxia or hypobaric hyperoxia may enhance vasodilatory function and lower effective systemic vascular resistance.

As used herein, the terms “treating,” “preventing” and “ameliorating” refer to interventions performed with the intention of alleviating the symptoms associated with, preventing the development of, or altering the pathology of a disease, disorder, or condition. Thus, in various embodiments, the terms may include the prevention (prophylaxis), moderation, reduction, or curing of a disease, disorder or condition at various stages. In various embodiments, therefore, those in need of therapy/treatment may include those already having the disease, disorder or condition and/or those prone to, or at risk of developing, the disease, disorder or condition and/or those in whom the disease, disorder or condition is to be prevented.

As used herein, the term “cardiovascular disease” refers to a class of diseases or disorders which involve the heart or blood vessels. The term is meant to include, but is not limited to, coronary artery disease, cerebrovascular disease, and peripheral artery disease. More particularly, the term is meant to include, but is not limited to, angina, myocardial infarction (heart attack), stroke, hypertensive heart disease, rheumatic heart disease, cardiomyopathy, heart arrhythmia, congenital heart disease, valvular heart disease, carditis, aortic aneurysms, subarachnoid hemorrhage, ischemia, cardiac insufficiency, atherosclerosis, hypertension; conditions associated with impaired blood circulation; and conditions where promotion of vasodilation may be beneficial. The cardiovascular disease may be acute or chronic. As used herein, the term “acute” refers to a cardiovascular disease of which the onset is sudden. As used herein, the term “chronic” refers to a cardiovascular disease which progresses slowly over time. In one embodiment, the cardiovascular disease comprises acute myocardial infarction (heart attack) and acute stroke.

As used herein, the term “subject” refers to any member of the animal kingdom. In one embodiment, the subject may be a human or other mammalian patient. Non-human subjects may include primates, livestock animals (e.g., sheep, cows, horses, goats, pigs) domestic companion animals (e.g., cats, dogs) laboratory test animals (e.g., mice, rats, guinea pigs, rabbits) or captive wild animals. In one embodiment, a subject is a human patient. In one embodiment, a subject is an adult patient. In one embodiment, a pediatric patient is a patient under 18 years of age, while an adult patient is a patient 18 years of age or older. In one embodiment, the subject may be one that exhibits one or more symptoms of cardiovascular disease. For example, the subject may have a history or family history of cardiovascular disease, or the subject may exhibit symptoms such as elevated blood pressure (i.e., hypertension), chest pain (angina), sudden numbness or weakness in the arms or legs, difficulty speaking or slurred speech, drooping muscles in the face, leg pain when walking, and/or claudication.

As used herein, the term “hypobaric” means having less than normal pressure, particularly pressure of an ambient gas being less than one atmosphere.

As used herein, the term “normoxia” means having a normal oxygen concentration expressed as an oxygen tension ranging between about 10% to about 21%.

As used herein, the term “oxygen tension” means the partial pressure of oxygen molecules dissolved in a liquid, such as blood plasma.

As used herein, the term “hyperoxia” means having an excess supply of oxygen expressed as an oxygen tension above about 21%.

As used herein, the term “vasodilation” or “vasodilatory function” means the widening, opening, or enlargement of a blood vessel (particularly the diameter of the interior or lumen of the blood vessel) as a result of relaxation in the smooth muscle cells with the vessel walls of arteries, and to a lesser extent, veins.

As used herein, the term “systemic vascular resistance” means the resistance of blood flow offered by all of the systemic vasculature, excluding the pulmonary vasculature. Systemic vascular resistance is determined by factors that influence vascular resistance in individual vascular beds. Mechanisms that cause vasoconstriction increase systemic vascular resistance, while mechanisms that cause vasodilation decrease systemic vascular resistance. Systemic vascular resistance is primarily determined by changes in blood vessel diameters.

In one embodiment, the invention comprises an apparatus or system for treating, preventing, or ameliorating cardiovascular disease, comprising:

an airtight chamber configured to accommodate and enclose a subject, and comprising at least one hatch to allow entry or exit of the subject;

a vacuum source for adjusting pressure within the chamber to a level sufficient to maintain hypobaria; and

a gas source for introducing one or more gases into the chamber, wherein the one or more gases comprises oxygen in an amount sufficient to produce a condition of either normoxia or hyperoxia within the chamber, while maintaining hypobaria.

Although the apparatus or system is capable of operating throughout a wide range of pressures, it is anticipated that most of the use of the apparatus or system would be performed under conditions of hypobaric normoxia or hypobaric hyperoxia. In one embodiment, the apparatus or system comprises a hypobaric normoxia apparatus or system, or a hypobaric hyperoxia apparatus or system.

It will be appreciated that either an apparatus or system are contemplated within the scope of the invention. In one embodiment, the apparatus may be constructed to include all features within a single unit; for example, an airtight chamber including its own vacuum source and a gas source integral to the chamber. In one embodiment, the system comprises all features as separate components which are operably connected to function as a whole.

The apparatus or system comprises an airtight chamber configured to accommodate and enclose a subject to be treated. The chamber is sufficiently shaped and sized to accommodate and enclose the subject. In one embodiment, the chamber is sufficiently shaped and sized to accommodate and enclose one or more human subjects in a standing, reclining, or seated position. In one embodiment, the chamber is sufficiently shaped and sized to accommodate and enclose one or more non-human subjects in a standing, reclining, or seated position for the purposes of veterinary medicine or animal research. In one embodiment, the chamber is sufficiently shaped and sized to accommodate and enclose one or more human or non-human subjects or both in a standing, reclining, or seated position.

It is contemplated that the shape, size, and exact dimensions of the chamber may vary, with such factors being dictated by the subject to be treated. In one embodiment, the chamber is cylindrical or rectangular-shaped. A more spacious, rectangular-shaped chamber which allows freedom of movement is considered less disconcerting or claustrophobic to the subject than the curved walls of a cylinder-shaped chamber. However, it will be appreciated by those skilled in the art that other shapes such as for example, triangular, square, and the like, are included within the scope of the invention.

In one embodiment, the chamber for use with a human subject is cylindrical-shaped, and has a length ranging from about 180 cm to about 300 cm, and a diameter ranging from about 50 cm to about 100 cm. In one embodiment, the chamber for use with a human subject is rectangular-shaped, and has a length ranging from about 180 cm to about 300 cm, a width ranging from about 180 cm to about 300 cm, and a height ranging from about 180 cm to about 300 cm.

In one embodiment, the chamber for use with a non-human subject is cylindrical-shaped, and has a length ranging from about 15 cm to about 45 cm, and a diameter ranging from about 15 cm to about 45 cm in diameter. In one embodiment, the chamber is cylindrical-shaped, and has a length of about 27 cm and a diameter of about 20 cm. In one embodiment, the chamber is rectangular-shaped, and has a length ranging from about 15 cm to about 45 cm, a width ranging from about 15 cm to about 45 cm, and a height ranging from about 5 cm to about 45 cm. In one embodiment, the chamber is rectangular-shaped, and has a length of about 27 cm, a width of about 15 cm, and a height of about 5 cm.

The chamber may be formed of various materials including, but not limited to, flexible, semi-rigid, or rigid materials. Such materials should be capable of withstanding reductions in pressure by vacuum. Suitable materials include, but are not limited to, poly(methyl methacrylate), vinyl, urethane-coated polyester, aluminum, steel, and the like. In one embodiment, the chamber is formed of clear poly(methyl methacrylate) (also known as Plexiglass™, Acrylite™, Lucite™, or Perspex™) to permit the subject to readily see outside the chamber to alleviate confinement anxiety, to allow technicians or medical staff outside the chamber to monitor the subject inside the chamber, and to illuminate the chamber interior with light. In one embodiment, the chamber is formed of opaque material and includes one or more view ports or windows formed of a clear material such as, for example, an acrylic plastic.

The chamber comprises at least one hatch to allow the subject to enter or exit the chamber. The hatch can be configured to allow entry or exit of medical equipment such as, for example, a wheelchair or a gurney. The hatch is movable between an open position wherein the subject may move or be moved into or out of the chamber, and a closed position, wherein the hatch forms a substantially air-tight seal against the chamber. In the closed position, the hatch and the chamber thus form an air-tight enclosure for the subject.

In one embodiment, the chamber comprises one or more pass-through ports to enable monitoring of the subject during treatment by allowing medical lines, electrical leads, cables, and the like to pass through the ports from one or more monitors or machines outside of the chamber to the subject within the chamber. It may be necessary, or at least desirable, to leave medical lines attached to the subject. As used herein, the term “medical line” means any tubing, wiring, and similar lines that are commonly connected to the subject including, but not limited to, lines for recording the heart rate, respiratory rate, blood pressure, temperature, and the amount of oxygen in the blood; for assisting in respiration (for example, an endotracheal tube); for feeding (for example, intravenous lines, umbilical catheter, oral and nasal feeding, central line), and the like. Multiple adhesive pads or cuffs may be placed upon the chest, legs, arms, and other body parts of the subject, and are connected by electrical leads to the respective monitors or machines.

In one embodiment, the chamber may be formed by renovating a hospital room to include the requisite adjustments including, but not limited to, strengthening of walls and/or windows to tolerate pressure differences, providing air tight seals and adequate vacuum technology to reduce the ambient pressure, and ensuring adequate ventilation and oxygen supplementation to avoid hypoxia within the room.

The apparatus or system further comprises a vacuum source for adjusting pressure within the chamber to a level sufficient to maintain hypobaria. The pressure is adjusted by extracting at least a portion of air within the chamber by vacuum. This effectively reduces the pressure within the chamber. In one embodiment, the pressure is adjusted to a pressure equivalent to the pressure encountered at an altitude between 1500 m and 3000 m above sea level. Either small (for example, 10 mmHg to 20 mmHg) or large (for example, 250 mmHg to 300 mmHg) reductions in pressure can be made as desired. In one embodiment, the pressure is adjusted to be lower than ambient air pressure by at least about 10 mmHg. In one embodiment, the pressure is adjusted to be lower than ambient air pressure by at least about 250 mmHg.

The types and operation of vacuum sources are commonly known to those skilled in the art and will not be discussed in detail. In one embodiment, the vacuum source comprises a vacuum pump. The vacuum pump can be any conventional device which can be connected to the chamber, and thereby provide reduced pressure within the chamber. The vacuum pump includes at least an internal or external power source, an air inlet port, and an exhaust outlet port. The air inlet port of the vacuum pump is connected to the chamber by suitable means (for example, a tube, hose, pipe, or the like) to evacuate air from within the chamber and to vent the removed air through the exhaust outlet port outside of the chamber. The vacuum pump is controlled by a vacuum controller in order to regulate the pressure within the chamber. In a laboratory setting, a suitable controller may comprise any conventional vacuum controller such as, for example, a Buchi™ Model V-850 or V-855 Vacuum Controller.

In one embodiment, the vacuum source comprises vacuum plumbing. Vacuum plumbing is commonly known to those skilled in the art as it is widely used in laboratories and healthcare facilities, and will not be discussed in detail. Vacuum plumbing comprises one or more lines or piping through which air passes as it is extracted from the chamber. Lines or piping may be installed or routed from walls or ceilings of a facility, and connected to the chamber to apply a vacuum to the chamber to extract at least a portion of air, thereby reducing the pressure within the chamber.

The apparatus or system further comprises a gas source for introducing one or more gases into the chamber. Gas sources are commonly known to those skilled in the art, and will not be discussed in detail. The gas source can be any conventional device or system which can be connected to the chamber, and thereby introduces or flows gas into the chamber. In one embodiment, the gas source comprises a gas intake system including a flow regulator for permitting or preventing gas flow into the chamber. In one embodiment, the gas source comprises gas plumbing. Gas plumbing is commonly known to those skilled in the art as it is widely used in laboratories and healthcare facilities, and will not be discussed in detail. Gas plumbing comprises one or more lines or piping through which gas passes as it is introduced or flowed into the chamber. Lines or piping may be installed or routed from walls or ceilings of a facility, and connected to the chamber to introduce or flow gas into the chamber.

In one embodiment, the gas comprises air. The air is used to ventilate the chamber, thereby preventing the build-up of carbon dioxide and excess humidity within the chamber. Air intake is balanced with the vacuum exhaust in order to ensure sufficient, stable ventilation of the chamber.

In one embodiment, the gas comprises oxygen. In one embodiment, oxygen is in the form of pure oxygen or oxygen-enriched air. It shall be noted that oxygen is not provided in the form of ambient air. In one embodiment, oxygen is introduced or flowed into the chamber in an amount sufficient to produce a condition of either normoxia or hyperoxia within the chamber, while maintaining hypobaria. Supplementation of oxygen within the chamber may be required when there is a substantial reduction in pressure (for example, a reduction of about 250 mmHg or about 300 mmHg). Reduction of pressure by vacuum can concomitantly lower the amount of oxygen within the chamber. The chamber is not intended to be utilized as a “hypoxic” chamber. As used herein, the term “hypoxia” means having a low oxygen concentration expressed as an oxygen tension ranging between about 1% to about 5%. Hypoxia may be deleterious for a subject suffering from severe cardiovascular disease. The level of oxygen is regulated within the chamber to ensure that the subject does not become hypoxic. In one embodiment, the pressure can be reduced sufficiently low such that the oxygen present within the chamber maintains a condition of normoxia, or supplemental oxygen can be introduced into the chamber to increase the amount of oxygen to produce a condition of normoxia. The conditions of hypobaria and normoxia are maintained concomitantly within the chamber in order to treat the subject properly. In one embodiment, supplemental oxygen can be introduced into the chamber to increase the amount of oxygen to produce a condition of hyperoxia. The conditions of hypobaria and hyperoxia are maintained concomitantly within the chamber in order to treat the subject properly.

In one embodiment, the apparatus or system comprises an alarm system comprising one or more sensors and one or more alarms. The alarm system can be any conventional alarm system which can be connected to the chamber, and thereby detect and respond to different parameters of interest within the chamber. In one embodiment, the sensors detect different parameters of interest within the chamber. Such parameters include, but are not limited to, pressure, the level of oxygen, and the level of carbon dioxide. In particular, the levels of oxygen and carbon dioxide must be maintained at safe levels within the chamber. The sensors transmit signals representative of the parameters to the alarm system. The alarm system may activate the alarms, warning lights, audible buzzers, or the like, for example, if the level of oxygen falls below or exceeds a predetermined threshold, or if the level of carbon dioxide exceeds a predetermined threshold.

Accordingly, the above described apparatus or system may be used to treat, prevent, or ameliorate cardiovascular disease in a subject. In one embodiment, the invention comprises use of an apparatus or system for treating, preventing, or ameliorating cardiovascular disease, the apparatus or system comprising:

an airtight chamber configured to accommodate and enclose a subject, and comprising at least one hatch to allow entry or exit of the subject;

a vacuum source for adjusting pressure within the chamber to a level sufficient to maintain hypobaria; and

a gas source for introducing one or more gases into the chamber, wherein the one or more gases comprises oxygen in an amount sufficient to produce a condition of either normoxia or hyperoxia within the chamber, while maintaining hypobaria.

In one embodiment, the invention comprises a method for treating, preventing, or ameliorating cardiovascular disease in a subject comprising exposing the subject for an effective duration to a condition of either hypobaric normoxia or hypobaric hyperoxia.

The detailed steps of the method are as follows. The hatch of the chamber is opened to allow a subject to be treated for cardiovascular disease to enter or to be moved (for example, by wheelchair or gurney) into the chamber. Within the chamber, the subject is placed comfortably into a standing position, or preferably in a reclining or seated position on a bed or chair. Any medical lines, electrical leads, cables, and the like for monitoring the subject are attached to the subject's body, routed through the pass-through ports to the outside of the chamber, and attached to respective monitors or machines outside of the chamber. The hatch is then closed to form a substantially air-tight seal against the chamber. Once closed, the hatch and the chamber thus form an air-tight enclosure for the subject.

The vacuum source is operated to extract at least a portion of air within the chamber, thereby adjusting the pressure to produce a condition of hypobaria within the chamber. The desired pressure is set and regulated using the vacuum controller. In one embodiment, the pressure is adjusted to a pressure equivalent to the pressure encountered at an altitude between 1500 m and 3000 m above sea level. In one embodiment, the pressure is adjusted to be lower than ambient air pressure by at least about 10 mmHg. In one embodiment, the pressure is adjusted to be lower than ambient air pressure by at least about 250 mmHg. In one embodiment, the pressure is adjusted to be lower than ambient air pressure in an amount sufficient to produce a condition of normoxia within the chamber, while maintaining hypobaria.

The gas source is operated to introduce one or more gases into the chamber. Air may be introduced into the chamber to ventilate the chamber in the event that carbon dioxide and excess humidity build-up within the chamber. Oxygen (for example, in the form of pure oxygen or oxygen-enriched air, but not ambient air) may be introduced into the chamber to prevent hypoxia within the chamber. In one embodiment, the oxygen is introduced in an amount sufficient to produce a condition of normoxia within the chamber, while maintaining hypobaria. In one embodiment, the oxygen is introduced in an amount sufficient to produce a condition of hyperoxia within the chamber, while maintaining hypobaria.

Treatments are administered to the subject under medical or technical supervision. The alarm system is operated in order to monitor, detect, and if necessary should an emergency situation arise, respond to different parameters such as, for example, the pressure, and levels of air, oxygen, and/or carbon dioxide. The sensors transmit signals representative of such parameters to the alarm system. The alarm system may activate the alarms, warning lights, audible buzzers, or the like, for example, if the level of oxygen falls below or exceeds a predetermined threshold, or if the level of carbon dioxide exceeds a predetermined threshold. The medical personnel or technician can thereby react by adjusting the vacuum or gas sources, or by removing the distressed subject from the chamber.

Treatments of acceptable duration can be administered. The duration of treatment depends upon many factors that are well known those skilled in the art, for example, the age, weight and general health condition of the subject; nature and extent of symptoms; any concurrent therapeutic treatments; frequency of treatment and the effect desired. As used herein, the term “effective duration” refers to a time period during which the subject is exposed to a reduced pressure within the chamber. An effective duration of treatment provides either subjective relief of symptoms or an objectively identifiable improvement as noted by the clinician or other qualified observer. In one embodiment, the duration of treatment is at least once daily for at least one hour. The number of sessions may vary depending upon the subject's response to treatment. The total number of sessions may range from at least one treatment to about fifty treatments.

In one embodiment, the method may be applied in conjunction with other types of treatments to the subject, e.g., to treat, prevent, or ameliorate cardiovascular disease. Non-limiting example of such treatments include any one or more of nitrates, alpha blockers, beta blockers, mixed alpha and beta blockers, calcium channel blockers, loop diuretics, thiazide diuretics, thiazide-like diuretics, potassium-sparing diuretics, dihydropyridines, non-dihydropyridines, ACE inhibitors, angiotensin II receptor antagonists, aldosterone receptor antagonists, vasodilators, alpha-2 agonists, adrenergic neuron blockers, or the like. These may occur for example, simultaneously or sequentially, in various embodiments.

In the development of the invention as described in the Examples, ex vivo evaluations of murine resistance arteries in a pressure myograph system were conducted. In resistance arteries perfused with physiologic pressures, acute exposure to reduced ambient air pressure enhanced both passive and active vasodilation with increasing perfusion pressure. A similar result was obtained when perfusion flow was increased as the independent variable. Exposure to reduced ambient air pressure increased flow-induced vasodilation. This phenomenon was preserved in the presence of nitric oxide and prostacyclin inhibitors, suggesting that it is not endothelium dependent. Similarly, methylcholine-induced vasodilation was also enhanced by acute exposure to reductions in ambient air pressure. These hypobaric-associated increases in vasodilation across a range of physiologic blood pressures increased effective arterial compliance.

In addition, in vivo evaluations of systemic hemodynamics using pressure volume catheters placed in the left ventricle were performed. In intact anaesthetized mice freely breathing oxygen, acute exposure to reduced ambient air pressure did not significantly alter blood pressure or myocardial contractility, but reduced systemic vascular resistance and increased cardiac output. Acute exposure to hypobaric pressures may increase effective arterial compliance, and reduce systemic vascular resistance and increase cardiac output in an intact circulation. Improving blood flow may have therapeutic implications for ischemic diseases like coronary artery disease (including acute coronary syndrome), cerebrovascular disease (including acute stroke), and peripheral artery disease. Reducing systemic vascular resistance and improving cardiac output may have therapeutic implications for heart failure, either from systolic dysfunction or heart failure with preserved ejection fraction.

Further, left anterior descending artery-ligation was used as a model for acute myocardial infarction. Following the operation, mice were subjected to low air pressure treatment. Compared to control mice, the treated mice exhibited significantly improved left ventricular function with respect to fractional shortening, ejection fraction, stroke volume, and cardiac output. These results indicate that low air pressure treatment may improve left ventricular function after myocardial infarction.

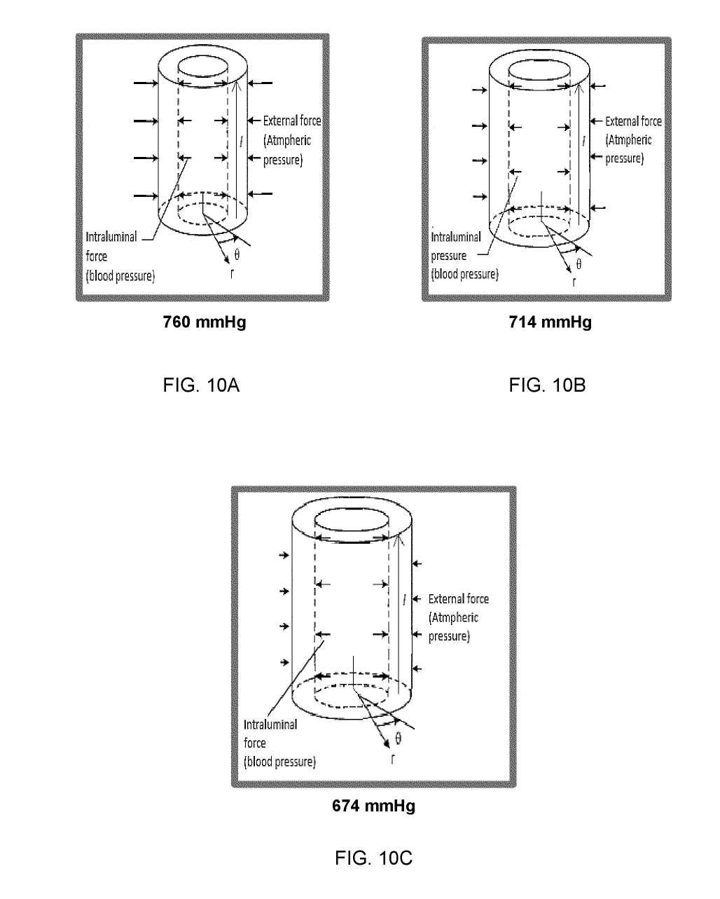

Without being bound by any theory, it is believe that lowering air pressure may generate a “pull” effect on the wall of the blood vessel as a non-invasive means of influencing myogenic tone to manipulate flow and resistance in the blood vessel. This “pull” effect may dilate the blood vessel, thereby obviating the need to introduce exogenous substances into the blood stream. Reduced compressive force on a blood vessel due to lowering air pressure (while interluminal pressure remains the same) may result in a reduced gradient of pressure across the vessel wall (FIGS. 10A-C). This may contribute to an increased tendency of the blood vessel to dilate upon exposure to lower air pressure.

Embodiments of the present invention are described in the following Examples, which are set forth to aid in the understanding of the invention, and should not be construed to limit in any way the scope of the invention as defined in the claims which follow thereafter.

### Example 1—Pressure Myograph System to Assess Vascular Function in Vitro

Four-month-old male C57 mice were given access to standard chow and water ad-libitum and were housed on a 12 h-12 h light-dark cycle. Mice were euthanized through sodium pentobarbital administered interperitonially, and their mesenteries were removed and placed in freshly prepared cold physiological salt solution (10 HEPES, 5.5 glucose, 1.56 CaCl₂, 4.7 KCl, 142 NaCl, 1.17 MgSO₄, 1.18 KH₂PO₄, pH 7.5.).

A resistance artery is small diameter blood vessel in the microcirculation that contributes significantly to the creation of the resistance to flow and regulation of the blood flow. Resistance arteries are usually arterioles or end-points of arteries. Having thick muscular walls and narrow lumen, they contribute the most to the resistance to blood flow. Degree of the contraction of muscles in the wall of a resistance artery is directly connected to the size of the lumen.

Two second order resistance arteries were dissected of surrounding connective tissue and mounted within a pressure myograph system (Living Systems Instrumentation; Burlington, Vt.). Vessels were tied onto glass cannulas with microscopic suture thread segments. Each vessel was immersed in a physiological salt solution bath, maintained at a temperature of 37° C. with the aid of a built-in temperature feedback mechanism connected to a miniature heating system. Intravascular pressure and flow were measured and alterable using pressure and flow control systems. A peristaltic pump was used to maintain specific rates of flow across the lumens vessels mounted in the pressure myograph system. To mimic physiological conditions as best as possible, the vessels were oriented as such that flow would be applied in the same direction as in vivo blood flow. The vessels were maintained at a perfusion pressure of 60 mmHg, the approximated in vivo mesentery arterial pressure of mice.

### Example 2—Drugs

Vessels were exposed to phenylephrine and methylcholine before the start of every protocol to ensure the vessel was intact and capable of responding to pressure and flow stimuli. In some protocols, one bath of the pressure myograph was infused with inhibitors of nitric oxide synthase and prostacyclins to inhibit the action of these two major endogenous endothelial vasodilators. This was performed in order to assess the effect of barometric pressure on mesenteric arteries isolated from the vasoactive influences of nitric oxide and prostacyclins.

### Example 3—Barometric Pressure Manipulations

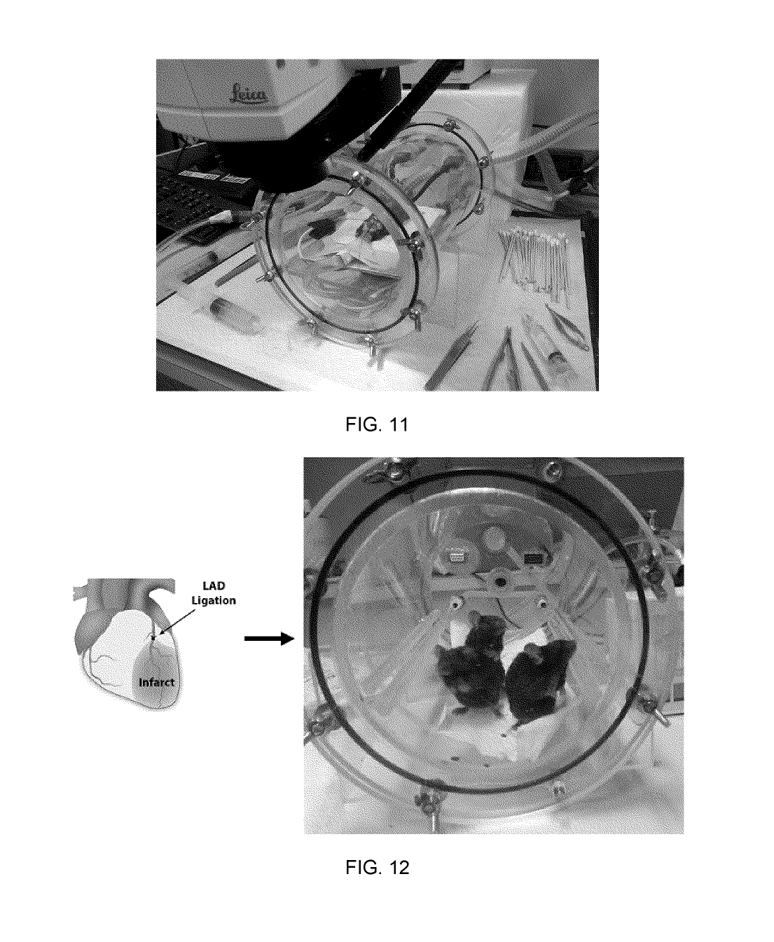

The entire myograph system was enclosed within a barometric pressure controlled chamber, capable of being sealed and pressure-controlled when necessary (FIG. 11). A control pressure was established on the pressure sensor in relation to the atmospheric pressure denoted by the meteorological service of Canada (Edmonton station). Pressure conditions PB714 and PB674 were assigned as 40 mmHg and 80 mmHg below atmospheric pressure, respectively. The conditions were maintained through a pressure apparatus.

A pressure transducer and flow regulator coupled with a peristaltic pump mechanisms was set up so as to be manipulated from outside upon sealing the barometric pressure chamber around the myograph system. Addition of any drugs in the myograph baths required opening of the chamber (and bringing the vessels back to room atmospheric pressure). Four barometric pressures were simulated: room atmospheric pressure (PB760), PB714, PB674, and a second baseline (PB760).

### Example 4—Measurements

Vessel diameters and wall thicknesses were recorded with the help of a microscope-coupled micrometer. Measurements were taken after every intervention, after the myograph had been sealed inside the barometric pressure controlled chamber. A see through top-lid allowed for micrometer measurements. Dessicants lined the inner walls of the chamber to ensure that visibility of the vessels was not diminished due to increasing humidity in the chamber.

### Example 5—Results

Discussed below are results obtained in connection with the experiments of Examples 1-4 which were performed to assess the responses of a mesenteric vessel to changes in barometric pressure.

i) Effect of Intraluminal Pressure Changed at Various Barometric Pressure Conditions (PB₇₆₀, PB₇₁₄, PB₆₇₄)

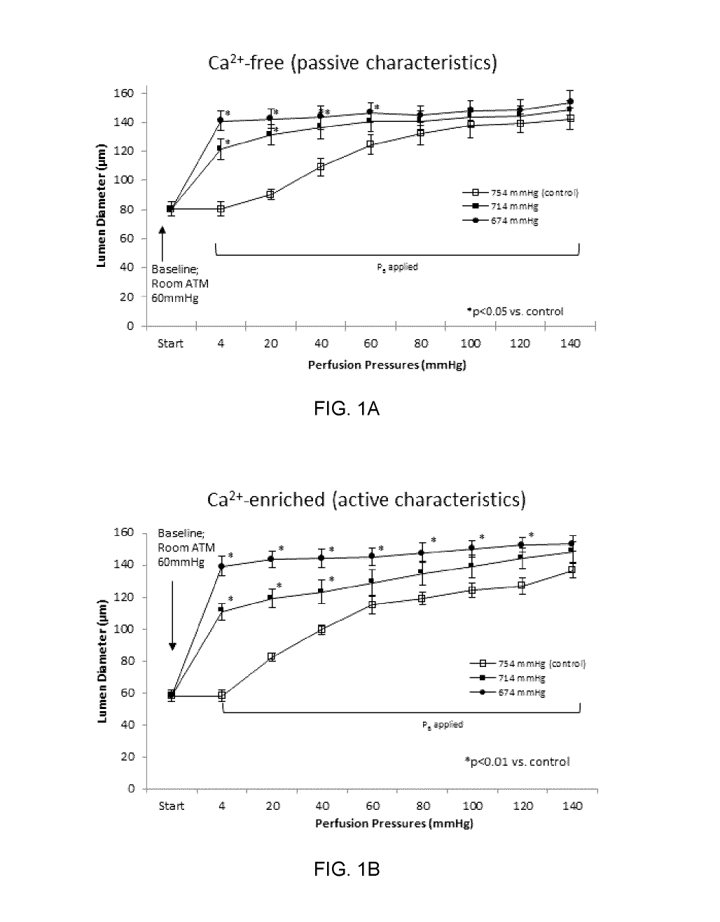

Mesenteric vessels hung and immersed in a Ca2+-free solution showed no constriction to phenylephrine or relaxation to methylcholine administered in the bath (n=7, p>0.05). Changes in vessel diameter were observed in response to manipulation in intraluminal pressure via a pressure transducer. FIG. 1A shows three perfusion pressures versus vessel lumen diameter (μm) curves, one at each barometric pressure condition (PB760, PB714, PB674). A maximal vessel dilation of 146 μm is reached at lower interluminal pressures when the barometric pressure is reduced (conditions of PB714, PB674). Myogenic tone is significantly reduced in vessels exhibiting physiological interluminal pressures (<60 mmHg) at barometric pressures below PB760 (n=7, p<0.05).

In parallel experiments, vessels immersed in a solution containing sufficient Ca2+(to mimic actual physiological conditions) also showed enhanced vasodilation, or reduced myogenic tone, in response to increasing interluminal pressures at reduced barometric pressures (FIG. 1B). Pressure curves were shifted upward from the room atmospheric pressure when the barometric pressure was lowered, with PB714 and PB674 acting additionally to dilate the vessels in response to intraluminal pressure increases (n=8, p<0.05) in comparison to PB760.

### ii) Flow-Mediated Vasodilation at Various Barometric Pressure Conditions (PB760, PB714, PB674)

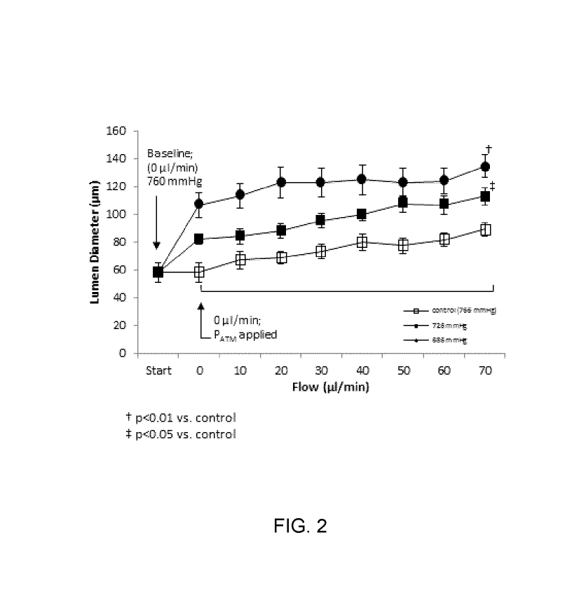

FIG. 2 shows changes in vessel lumen diameter (μm) to step-wise increases in flow (μl/min) observed in the vessel. Vessels at lower barometric pressures (PB714, PB674) have significantly larger lumen diameters at the condition of 0 μl/min flow in comparison to PB760 at 0 μl/min (n=9, p<0.001). As the flow is increased stepwise, vessels in all barometric pressure conditions react with dilation. However, the greatest dilation (or most responsiveness) to increases in flow is seen in vessels existing in a barometric pressure PB674 in relation to PB760 (n=8, p<0.001).

iii) Effect of Prostaglandins and Nitric Oxide Synthase on Vessel Responses to Step-Wise Increases in Intraluminal Pressure and Methylcholine Addition Under Barometric Pressure Conditions (PB760, PB714 and PB674)

Inhibitors, namely meclofenamate and L-N^{G}-Nitroarginine methyl ester (L-NAME), were used to inhibit the action of prostacyclins and nitric oxide synthase in order to assess the effect of barometric pressure on mesenteric arteries isolated from the vasoactive influences of nitric oxide and prostacyclins.

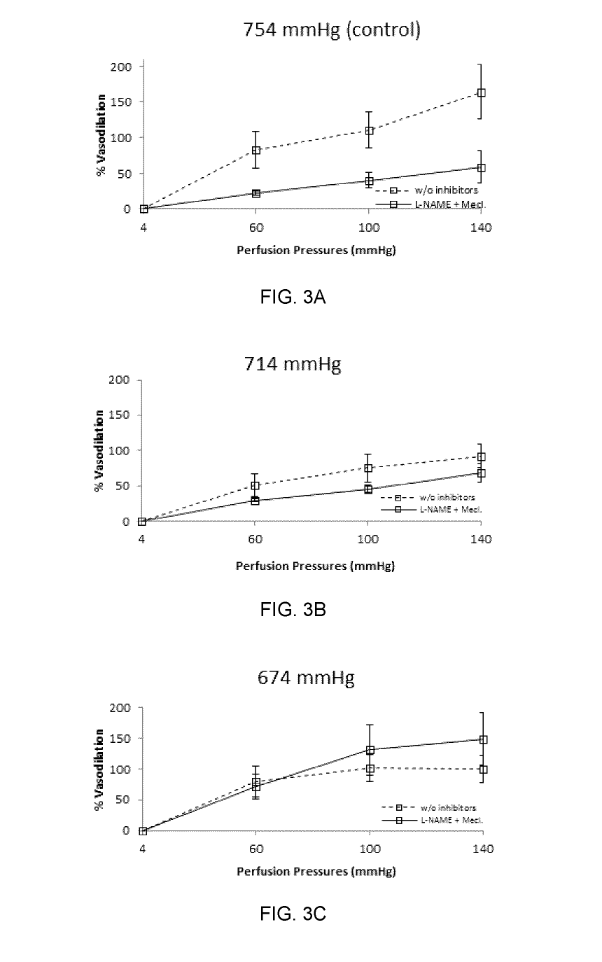

Mesenteric vessels immersed in a bath of physiological salt solution with added meclofenamate and L-NAME at lower atmospheric pressures (PB714, PB674) showed similar responses to changes in interluminal pressure (FIGS. 3A-C) as vessels with no inhibitors (p>0.05). Significant lumen dilation to reduced barometric pressure was observed even in the presence of L-NAME and meclofenamate.

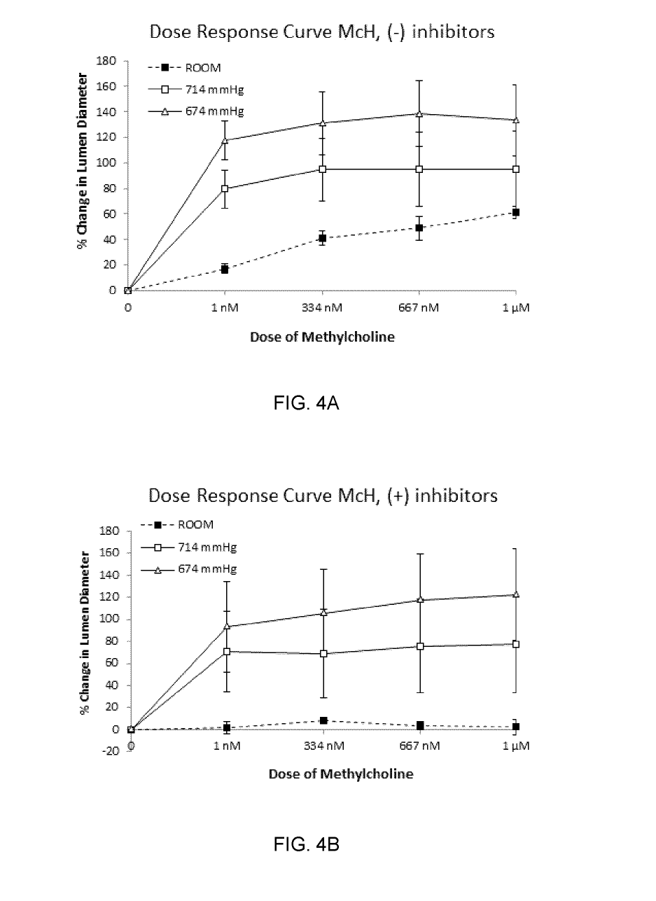

Methycholine is a drug used to cause dilation of blood vessels. In a cumulative response curve to methylcholine (doses ranging from 1 nM to 1 μM), vessels with inhibitors added showed a retained response to reduced barometric pressure, as shown by a maximal 122% increase in lumen diameter at PB674 without inhibitors (FIG. 4A) compared to 133% increase at PB674 with L-NAME and meclofenamate (FIG. 4B).

### Example 6—In Vivo Experiment

Left ventricular catheterization procedures were completed on male C57 mice (aged 4-6 months old) using a catheter (SciScence™) inserted via the carotid artery into the left ventricle. The surgery was performed under 2.0% isoflourane anesthesia. The operation on the mice was performed inside a custom constructed chamber, sealable after catheter insertion to regulate barometric pressure (FIG. 12). LabChart 3™ provided real time data and derived pressure volume loops from a number of measured and calculated hemodynamic variables. The barometric pressure inside the chamber was recorded for five minutes at four pressure conditions (PB760, PB714, PB674, and PB760) before the catheter was removed and the animal was cervically dislocated. Pressure-time relationships were recorded in the aorta before advancing the catheter through the aortic valve into the left ventricle.

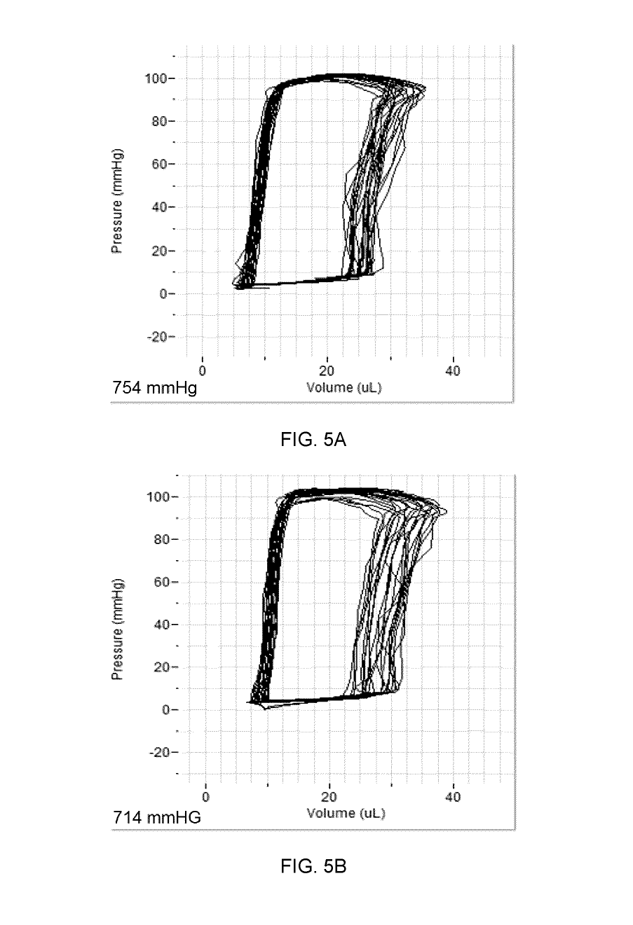

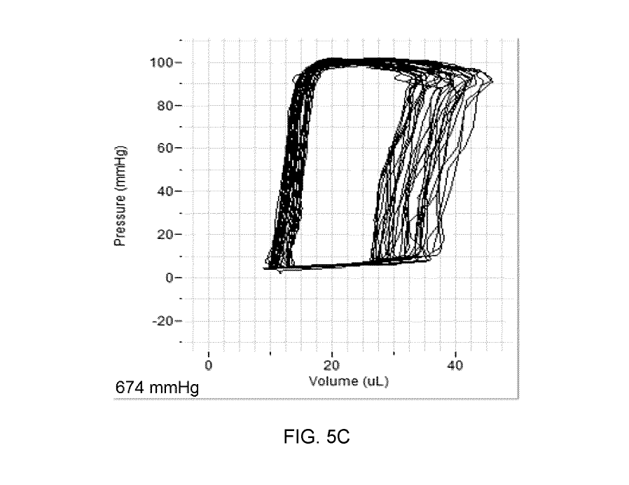

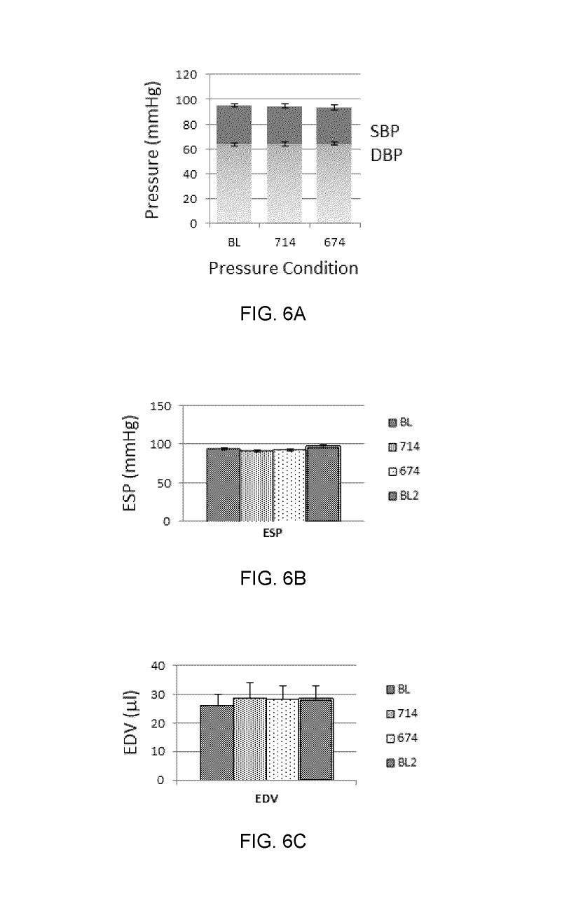

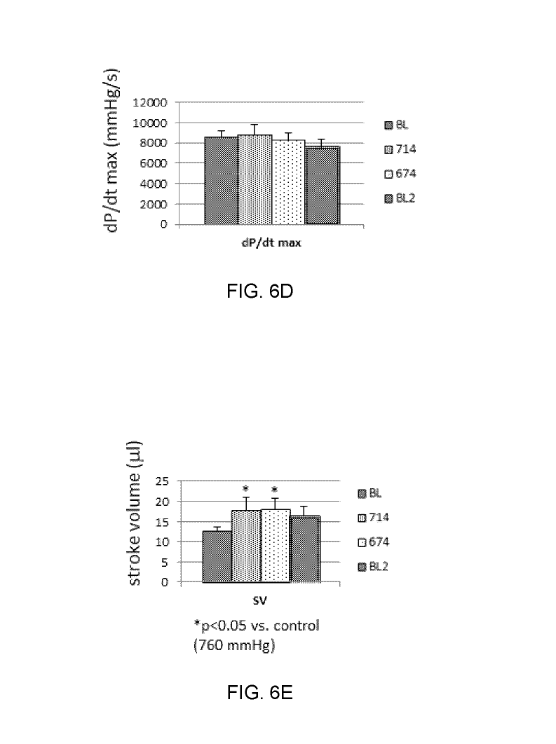

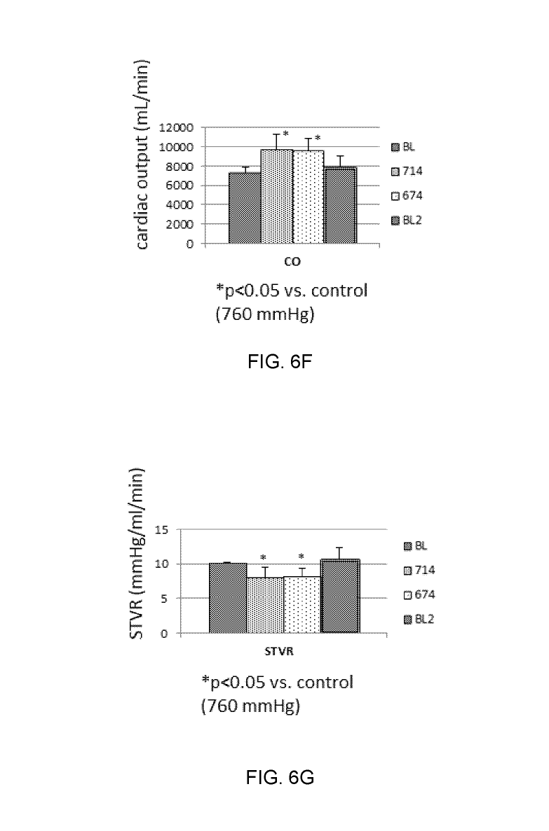

FIGS. 5A-C are graphs showing left ventricular pressure-volume relationships expressed as PV loops obtained via invasive catheterization of the left ventricle through the carotid artery in a closed chest procedure. The Transonic™ software coupled with the SciScence™ catheter calculated and displayed the parameters showed in FIGS. 6A-G in real time. FIG. 6A shows the systolic and diastolic blood pressures at each of the barometric pressure conditions PB760, PB714, and PB674. Invasive pressure-volume hemodynamic analyses showed preserved end-systolic pressure (ESP, FIG. 6B), end-systolic volume (EDV, FIG. 6C), and maximum derivative of change in systolic pressure over time (dp/dt max) across the barometric pressure conditions PB760, PB714, and PB674 (FIG. 6D). Stroke volume (FIG. 6E) and cardiac output (FIG. 6F) were increased significantly (n=9, p<0.05) at PB674 and PB714 as compared to PB760. The systemic total vascular resistance (STVR, FIG. 6G) was decreased significantly at PB674 and PB714 as compared to PB760. Stroke volume and cardiac output indices returned to near baseline values when pressures were returned to PB760 at the end of each acute protocol. The mean arterial pressure, measured with the catheter pulled back into the aorta, was not different among the barometric pressure conditions.

### Example 7—Use of Low Air Pressure to Treat Myocardial Infarction in Mice In Vivo

Left anterior descending artery (LAD)-ligation was used as a model for acute myocardial infarction. The LAD is permanently ligated with one single stitch, forming an ischemia that can be observed almost immediately. By closing the LAD, no further blood flow is permitted in that area, while the surrounding myocardial tissue is nearly not affected. This surgical procedure imitates the pathobiological and pathophysiological aspects occurring in infarction-related myocardial ischemia.

LAD-ligation was performed on three-month old C57BL6 male mice. Control mice (n=9) were allowed to recover from the surgery at atmospheric pressure (754 mmHg). Treated mice (n=8) were placed in a hypobaric chamber (i.e., subjected to low air pressure, FIG. 12) to recover from the LAD-ligation for three hours at 714 mmHg, a pressure chosen to mimic an elevation of 1500 m and avoid hypoxemia. The successful induction of anterior myocardial infarction was confirmed by echocardiography twenty-four hours after the surgery. The low air pressure treated mice were administered at least three hours of hypobaric treatment daily for seven days. Echocardiographic evaluation of left ventricular (LV) function was performed for all mice after seven days.

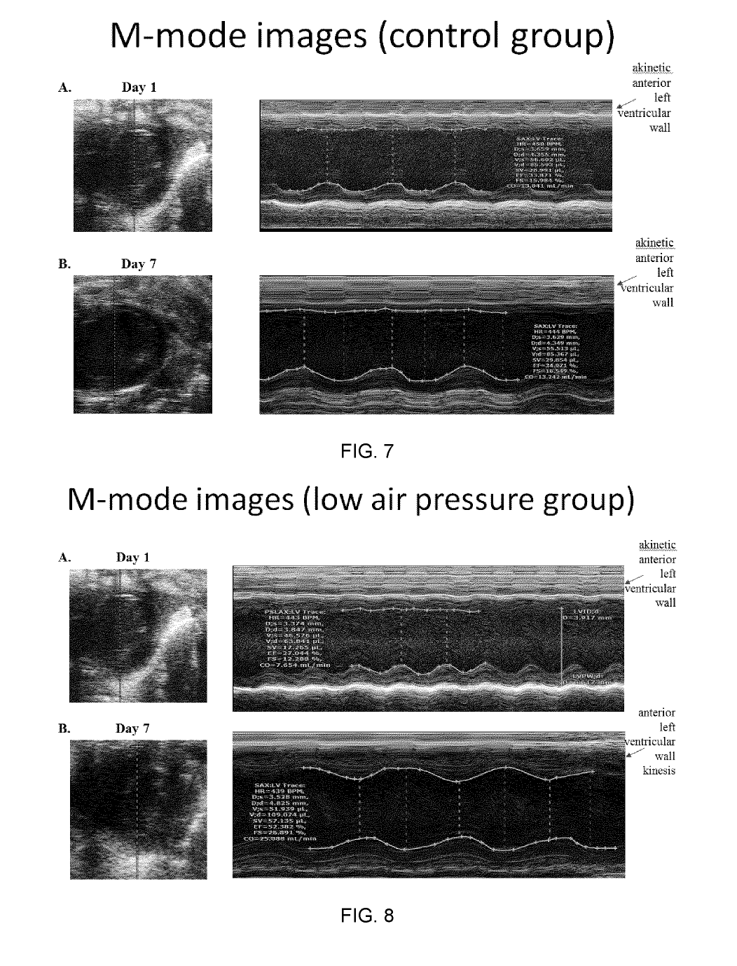

Transthoracic images were obtained on anesthetized control mice on Day 1 (immediately after LAD-ligation surgery) and on Day 7 (FIG. 7). M-mode images were captured using a parasternal short axis view. On Day 1, the anterior walls were observed to be akinetic on M-mode images, consistent with a large anterior acute myocardial infarction (panel A). On Day 7, the M-mode images demonstrated no improvement in anterior wall function, with the anterior walls observed to be akinetic (panel B).

Transthoracic images were obtained on anesthetized low air pressure treatment mice also on Day 1 (immediately after LAD ligation surgery) and on Day 7 (FIG. 8). M-mode images were captured using a parasternal short axis view. On Day 1, the anterior walls were observed to be akinetic on M-mode images, consistent with a large anterior acute myocardial infarction (panel A). On Day 7, the M-mode images demonstrated improvement in anterior wall function. The anterior walls were observed to be moving more, consistent with better left ventricular function after seven days of low air pressure treatment (panel B).

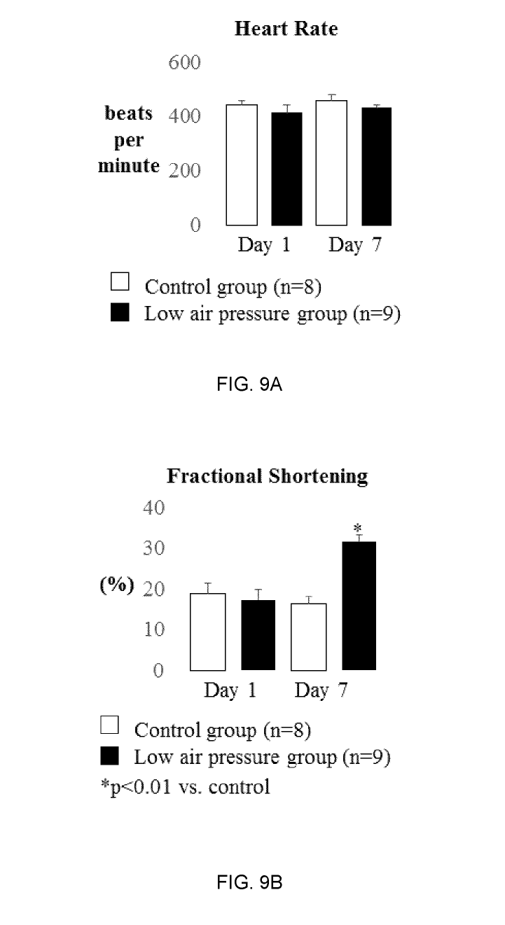

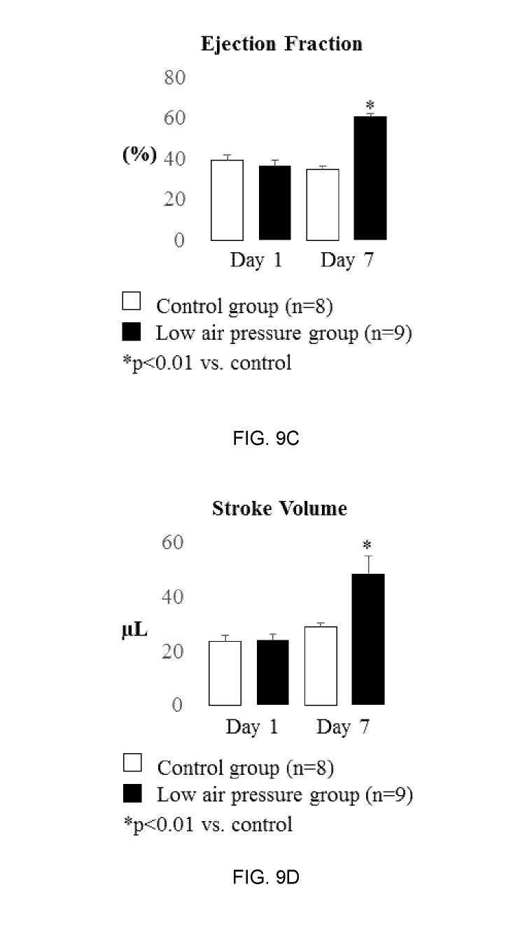

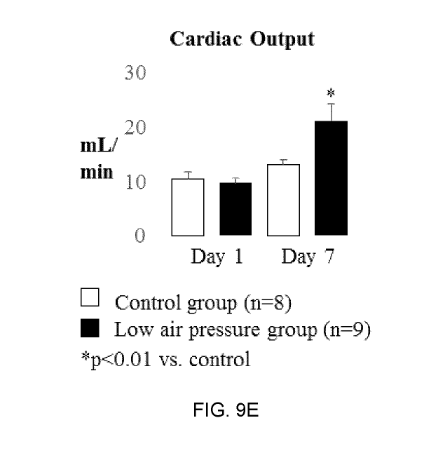

After seven days of low air pressure treatment, the mice had significantly improved left ventricular function, while control mice did not (FIGS. 9A-E). The heart rates of control and treated mice were not different (FIG. 9A). Fractional shortening (i.e., the degree of shortening of the left ventricular diameter between end-diastole and end-systole) was similar between control and low air pressure treated mice on Day 1. However, on Day 7, there was a significant increase in fractional shortening in low air pressure treated mice, consistent with better left ventricular function (FIG. 9B). Similarly, left ventricular ejection fraction (i.e., measure of percentage of blood leaving the heart each time it contracts) (FIG. 9C), stroke volume (i.e., amount of blood ejected by the left ventricle in one contraction) (FIG. 9D), and cardiac output (i.e., the amount of blood put out by the left ventricle of the heart in one contraction) (FIG. 9E) were all improved in the low air pressure treated mice on Day 7. These results indicate that low air pressure treatment may improve left ventricular function after myocardial infarction.

### REFERENCES

All publications mentioned are incorporated herein by reference (where permitted) to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
Burtscher, M. (2014) Effects of living at higher altitudes on mortality: a narrative review. Aging. Dis. 2014 Aug. 5(4):274-280.
Shahid, A. and McMurtry, M. S. (May 14, 2015) Acute changes in ambient air pressure modulate vasodilation of resistance arteries independently of endothelial mechanisms. Poster presented at Research Day 2015, Department of Medicine, University of Alberta, Edmonton, Alberta, Canada.
Shahid, A., McMurtry, M. S., Patel, V., Morton, J. S., Davidge, S. T. and Oudit, G. (Jun. 8, 2015) Acute reduction of ambient air pressure augments effective arterial compliance, enhancing vasodilation and lowering systemic vascular resistance in vivo. Poster presented to the Department of Medicine, University of Alberta, Edmonton, Alberta, Canada.
Shahid, A., Morton, J. S., Davidge, S. T. and McMurtry, M. S. (April 2016) Acute reduction of ambient air pressure enhances arterial vasodilation in murine resistance arteries independently of endothelial factors. Abstract presented at Experimental Biology 2016 Meeting. Published in The FASEB Journal 30(1): Supplement 947.8.

1. A method for treating, preventing, or ameliorating cardiovascular disease in a subject comprising exposing the subject for an effective duration to a condition of either hypobaric normoxia or hypobaric hyperoxia.

2. The method of claim 1, comprising enclosing the subject within an airtight chamber wherein pressure within the chamber is adjusted to maintain hypobaria.

3. The method of claim 2, comprising adjusting the pressure by extracting at least a portion of air within the chamber.

4. The method of claim 3, wherein the pressure is adjusted to a pressure equivalent to the pressure encountered at an altitude between 1500 m and 3000 m above sea level.

5. The method of claim 3, wherein the pressure is adjusted to be lower than ambient air pressure by at least about 10 mmHg.

6. The method of claim 3, wherein the pressure is adjusted to be lower than ambient air pressure by at least about 250 mmHg.

7. The method of claim 3, wherein the pressure is adjusted to be lower than ambient air pressure in an amount sufficient to produce a condition of normoxia within the chamber, while maintaining hypobaria.

8. The method of claim 3, further comprising introducing air into the chamber to ventilate the chamber.

9. The method of claim 3, further comprising introducing oxygen into the chamber to prevent hypoxia at the adjusted pressure.

10. The method of claim 9, wherein the oxygen is introduced in an amount sufficient to produce a condition of normoxia within the chamber, while maintaining hypobaria.

11. The method of claim 9, wherein the oxygen is introduced in an amount sufficient to produce a condition of hyperoxia within the chamber, while maintaining hypobaria.

12. The method of claim 9, wherein the oxygen is in the form of pure oxygen or oxygen-enriched air.

13. The method of claim 1, wherein the cardiovascular disease is selected from coronary artery disease, cerebrovascular disease, or peripheral artery disease.

14. The method of claim 1, wherein the cardiovascular disease is selected from myocardial infarction or stroke.

15. The method of claim 1, wherein the effective duration is at least once daily for at least about one hour.

16-24. (canceled)

25. An apparatus or system for treating, preventing, or ameliorating cardiovascular disease, comprising:
an airtight chamber configured to accommodate and enclose a subject, and comprising at least one hatch to allow entry or exit of the subject;
a vacuum source for adjusting pressure within the chamber to a level sufficient to maintain hypobaria; and
a gas source for introducing one or more gases into the chamber, wherein the one or more gases comprises oxygen in an amount sufficient to produce a condition of either normoxia or hyperoxia within the chamber, while maintaining hypobaria.

26. The apparatus or system of claim 25, further comprising an alarm system comprising one or more sensors and one or more alarms.
